(19) United States
(12) Patent Application Publication
(10) Pub. No.: US 2017/0368343 A1
(43) Pub. Date: Dec. 28, 2017

(54) MEMORIES ALIVE

(71) Applicant: Memories Alive, LLC, Dallas, TX (US)

(72) Inventors: Paula Marie Jones, Dallas, TX (US); Jeffrey Griffin Jones, Dallas, TX (US); Saba Ahmed, Garland, TX (US); Hood Harcourt Whitson, Fort Worth, TX (US); Lauren Dunlap Whitson, Fort Worth, TX (US); Brian Scott Youngblood, Dallas, TX (US)

(73) Assignee: Memories Alive, LLC

(21) Appl. No.: 15/630,730

(22) Filed: Jun. 22, 2017

Related U.S. Application Data
(60) Provisional application No. 62/353,397, filed on Jun. 22, 2016.

(51) Int. Cl. A61N 1/36; A61B 5/0482; A61N 1/00

(52) U.S. Cl. CPC A61N 1/36014; A61N 1/36082; A61B 5/0482; A61N 1/36025; A61N 1/00

(57) ABSTRACT

The disclosed embodiments include systems and methods to provide a patient suffering from at least one communication and memory impairment with positive stimuli. In one of such embodiments, the method includes obtaining data indicative of a current condition of a first patient. The method also includes determining, based on prior patient data of the first patient, a first stimulus that triggered a positive response from the first patient while the first patient was in a condition similar to the current condition. The method further includes providing a first recommendation for the first patient to experience the first stimulus for display on an electronic device. The method further includes receiving a first response triggered by the first stimulus. The method further includes storing data indicative of the first response in a storage medium.

### BACKGROUND

The present disclosure relates generally to systems and methods to improve physical and psychological conditions of a patient who suffers from communication and/or memory impairment.

Millions of individuals around the world suffer from communication and/or memory impairments caused by various neurodegenerative diseases such as Alzheimer's and other dementias, post-traumatic stress disorder (“PTSD”), traumatic brain injury, stroke, chronic traumatic encephalopathy, autism, as well as other conditions that adversely affect communication and/or mental capacities of said individuals. Despite vast technological and medical advances, cures for diseases and/or disorders that cause many of the foregoing communication and memory impairments remain elusive. As such, millions of individuals experience conditions attributed to and/or caused by different communication and/or memory impairments with little means to improve said impairments.

### BRIEF DESCRIPTION OF THE DRAWINGS

Illustrative embodiments of the present invention are described in detail below with reference to the attached drawing Figures, which are incorporated by reference herein, and wherein:

FIG. 1 is a network environment for providing positive stimuli to a patient suffering from a communication and/or memory impairment in accordance with one embodiment.

FIG. 2 is a flow chart that illustrates a process to provide positive stimuli to a patient suffering from a communication and/or memory impairment in accordance with one embodiment.

FIG. 3A is a flow chart that illustrates a process to dynamically formulate new stimuli based on the patient's responses to stimuli in accordance with one embodiment.

FIG. 3B is a flow chart that illustrates a process to dynamically formulate new stimuli based on the patient's responses to stimuli in accordance with another embodiment.

The illustrated figures are only exemplary and are not intended to assert or imply any limitation with regard to the environment, architecture, design, or process in which different embodiments may be implemented.

### DETAILED DESCRIPTION

In the following detailed description of the illustrative embodiments, reference is made to the accompanying drawings that form a part hereof. These embodiments are described in sufficient detail to enable those skilled in the art to practice the invention, and it is understood that other embodiments may be utilized and that logical structural, mechanical, and electrical, changes may be made without departing from the spirit or scope of the invention. To avoid detail not necessary to enable those skilled in the art to practice the embodiments described herein, the description may omit certain information known to those skilled in the art. The following detailed description is, therefore, not to be taken in a limiting sense, and the scope of the illustrative embodiments is defined only by the appended claims.

The present disclosure relates to systems and methods to provide a patient suffering from an adverse mental condition with positive stimuli. In one embodiment, a software and/or firmware application (“application”) running on an electronic device is operable to obtain data indicative of a current condition of a patient. The current condition of the patient may be based on a combination of the patient's physical conditions and psychological conditions as determined by sensors that are operable to monitor one or more physical and/or physiological conditions of the patient. The current condition may also be determined by the patient's caregiver (e.g., nurse, doctor, family member). Examples of the patient's physical condition include the patient's heart rate, blood pressure, oxygen level, breathing patterns, glucose level, cortisol, pedometer, endorphins, appetite, weight, reliance on medication, urinary output as well as other assessable physical conditions of the patient. Further, examples of the patient's psychological conditions include the patient's mood, anxiety, self-awareness, cognizance, responsiveness, as well as other assessable psychological conditions of the patient.

The application then assesses prior patient data of the patient to identify stimulus previously experienced by the patient that are triggered a positive response from the patient. A stimulus may be associated with a text, an image, a sound, a recording, or a type of multimedia content the patient has previously experienced. For example, the stimulus may be an artwork painted by the patient, a song composed by the patient, an image of the patient, a video recording of the patient's children, as well as other experiences of the patient that are captured by text, image, sound, video recording, or another mechanism. Further, the stimulus may also be associated with an activity, such as a dance routine, a game, a sing-along, or another activity the patient has previously participated in or has experienced. As defined herein a positive response is a response that immediately improves the patient's condition, improves the patient's condition over short duration, and/or improves the patient's condition over a prolonged duration. The stimulus may trigger an immediate positive response, such as a positive change in the patient's mood, an improvement in conversational skills, or other positive responses that are noticeable immediately or almost immediately after the patient has experienced the stimulus. The stimulus may also trigger a positive response that occurs at a later time, such as an improvement in sleeping pattern, an improvement in short/long term memory, a decrease in medication intake, an increase in daily grooming, a reduction in fear-associated behaviors, a reduction in anxiety-associated behaviors, a reduction in assistance from caregivers, an increase in social interactions, as well as other positive responses that may occur within a period of time after the patient experiences the stimulus. In some circumstances, the stimulus may also trigger a negative response, a neutral response, or no response from the patient. The application is operable to categorize each response into one the foregoing types of responses and to determine why the stimulus generated a certain type of response and to limit future recommendations to positive responses.

The application then provides a recommendation for the patient to experience the stimulus. In some embodiments, the recommendation is provided for display on the same electronic device the application operates on. In other embodiments, the recommendation is provided for display on another electronic device that is deployed within proximity of the patient. In further embodiments, the recommendation is communicated to the caregiver. The recommendation may also include instructions to the caregiver on how the stimulus should be presented to the patient.

The patient's response to the stimulus is monitored and assessed. In some embodiments, the patient's response is automatically monitored by the sensors that are connected to the patient and data indicative of the patient's response are provided to the application. In other embodiments, the patient's response is manually monitored by the caregiver and data recorded by the caregiver and indicative of the patient's response are provided to the application. Data indicative of the patient's response are then stored on a storage medium containing prior patient data of the patient.

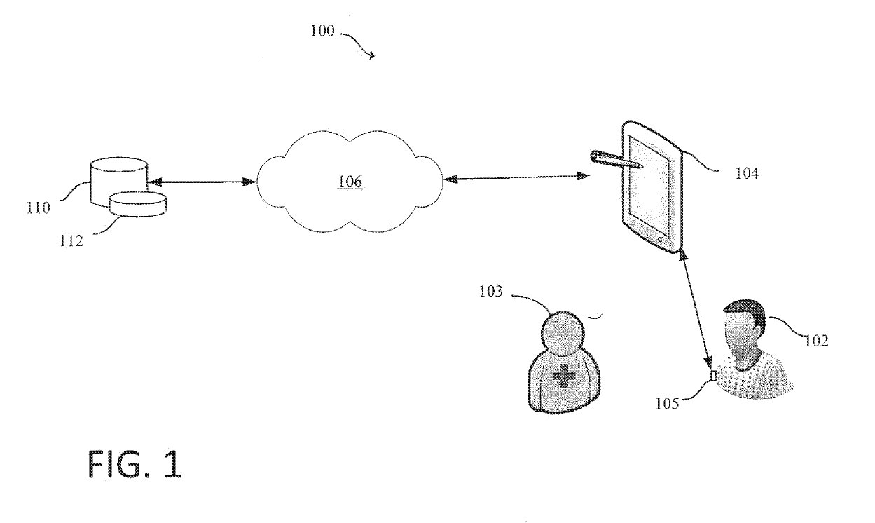

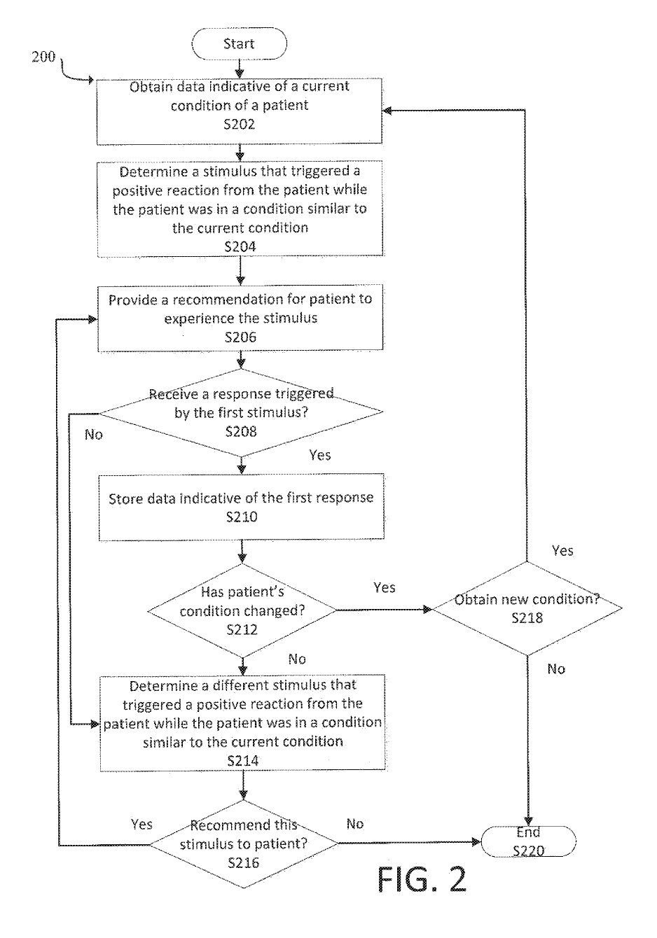

The application may continuously and/or periodically provide recommendations of additional stimulus to the patient to improve the patient's condition. The application may also formulate new stimulus based on the patient's current condition as well as the patient's responses to stimulus provided to the patient. Moreover, the application may compare the stimulus with one or more similar stimuli to determine why the stimulus triggered a particular response from the patient. The application may also formulate new stimuli to trigger more positive responses from the patient. As such, the application may dynamically learn from the patient's recent behaviors to become better tailored towards the patient's conditions, and dynamically provide stimuli that trigger positive responses from the patient. Additional details of systems and methods for providing the patient with positive stimuli are provided in the paragraphs below and are illustrated in at least FIGS. 1 and 2.

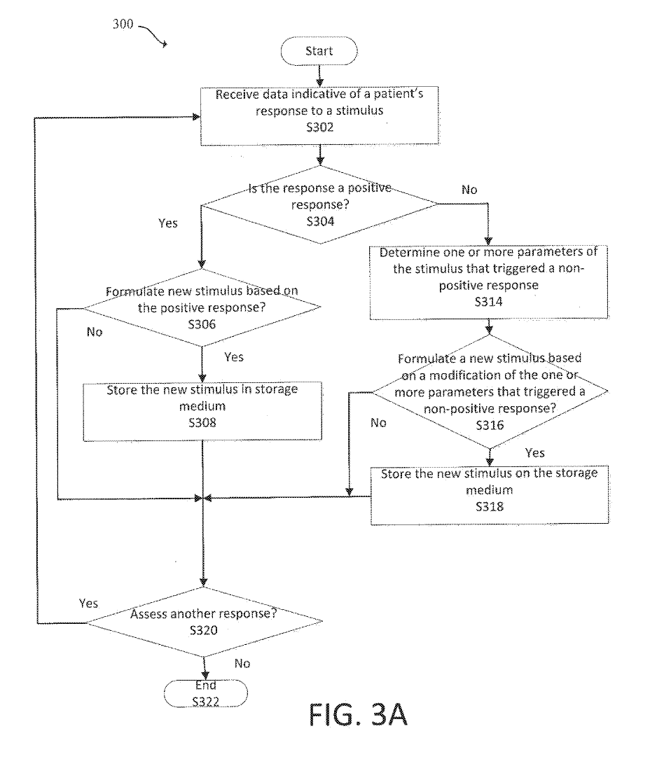

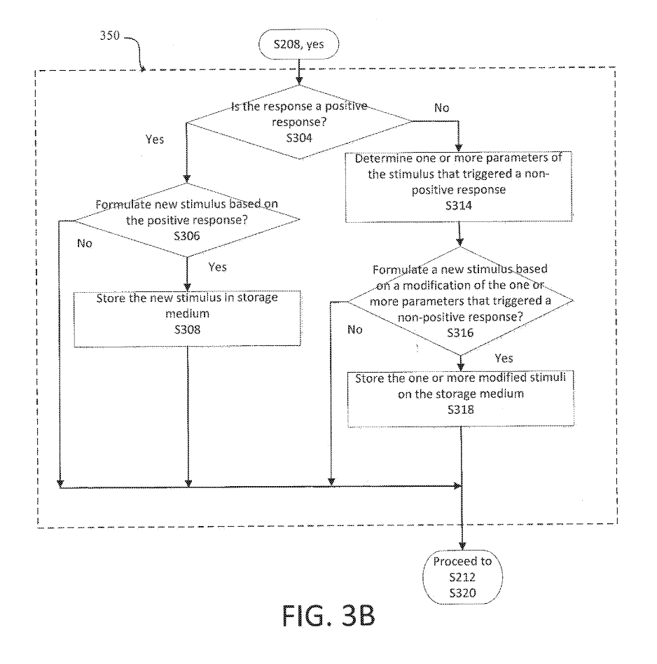

Now turning to the figures, FIG. 1 is a network environment 100 for providing a patient suffering from a communication and/or a memory impairment with positive stimuli in accordance with one embodiment. The network environment 100 includes a first electronic device 104 placed within proximity of a patient 102 and a caregiver 103. As defined herein, the caregiver 103 includes any individual who operates the first electronic device 104 to provide one or more stimuli to the patient 102. Examples of the caregiver 103 include doctors, nurses, acquaintances, friends, relatives, as well as other individuals authorized to operate the first electronic device 104. The first electronic device 104 includes any electronic device operable to obtain data indicative of a current condition of the patient 102, determine which stimulus the patient should experience to trigger a positive response from the patient, provide the stimulus for display, and receive data indicative of the patient's response to the stimulus. Although FIG. 1 illustrates the first electronic device 104 as a tablet computer, the first electronic device 104 may also be implanted as a smartphone, laptop computer, desktop computer, smart television, electronic watch, PDA, as well as similar electronic devices having hardware, software, and/or firmware that are operable to perform the operations described in the foregoing sentences. Additional operations performed by the first electronic device 104 are provided herein and are illustrated in at least FIGS. 2, 3A, and 3B.

The network environment 100 further includes a storage medium 110 that is communicatively connected to the first electronic device 104 via network 106. The storage medium 110 may be formed from data storage components such as, but not limited to, read-only memory (ROM), random access memory (RAM), flash memory, magnetic hard drives, solid state hard drives, CD-ROM drives, DVD drives, floppy disk drives, as well as other types of data storage components and devices. In some embodiments, the storage medium 110 includes multiple data storage devices. In further embodiments, the multiple data storage devices may be physically stored at different locations. In one of such embodiments, the data storage devices are components of a server station, such as a cloud server. In another one of such embodiments, the data storage devices are components of a local management station of a facility the patient 102 is staying at. The storage medium 110 includes data indicative of a history of the patient's prior conditions, stimuli previously experienced by the patient, as well as the patient's responses to said stimuli. In some embodiments, the storage medium 110 also includes instructions to determine which stimulus the patient should experience to trigger positive responses from the patient, instructions to provide the stimulus for display, instructions to receive data indicative of the patient's response to the stimulus, as well as other instructions described herein to provide the patient 102 with positive stimuli.

In some embodiments, the storage medium 110 further includes a behavior databank 112. The behavior databank 112 contains a compilation of analytical and quantitative data indicative of multiple patients' prior conditions, stimuli experienced by said patients, and said patients' responses to said stimuli. In one of such embodiments, data stored in the behavior databank 112 are categorized by patient condition, stimulus, and patient response to a stimulus. Moreover, the behavior databank 112 contains a compilation of stimuli presented to patients suffering from a condition. For example, the behavior databank 112 includes data indicative of patients that experienced a condition characterized by high blood pressure and heart rate of 100-110 (hereafter referred to as the “first general condition”). Further, the behavior databank 112 also includes data indicative of different stimuli disclosed herein that were presented to patients that experienced the first general condition, as well as responses of the patients that experienced the first general condition. Continuing with the foregoing example, the behavior databank 112 includes data that indicate a first general condition of disorientation was experienced 1,000times, a picture of a patient with a grandchild was presented 800 times in response to the first general condition and triggered 600 positive reactions, a video of a patient with family at a holiday was presented 750 times and triggered 700 positive reactions, and a song associated with a patient's childhood was played 600 times and triggered 300 positive reactions. The first electronic device 104 is operable to access the behavior databank 112 to obtain analytical and quantitative data stored in the behavior databank 112, analyze said analytical and quantitative data, and provide recommendations based on said analytical and quantitative data. Additional descriptions of operations performed by the first electronic device 104 to retrieve analytical and quantitative data from the behavior database, assess said analytical and quantitative data, and provide recommendations based on said analytical and quantitative data are provided in the below paragraphs. In one of such embodiments, data stored in the behavior databank 112 are not associated with patient identities. As such, while the behavior databank 112 contains data indicative of conditions of different patients associated with categories of stimulus with a high probability of improving such condition, the data does not identify which patients experienced said conditions. Although the storage medium 110 of FIG. 1 is illustrated to be stationed at a remote location relative to the first electronic device, in some embodiments, the storage medium 110 is stationed at a local location relative to the first electronic device 104, or is a subcomponent (e.g., an internal and/or external storage medium) of the first electronic device 104.

The network 106 can include, for example, any one or more of a cellular network, a satellite network, a personal area network (PAN), a local area network (LAN), a wide area network (WAN), a broadband network (BBN), a RFID network, a Bluetooth network, a device to device network, the Internet, and the like. Further, the network 106 can include, but is not limited to, any one or more of the following network topologies, including a bus network, a star network, a ring network, a mesh network, a star-bus network, tree or hierarchical network, or similar network architecture. The network 106 may be implemented using different protocols of the internet protocol suite such as TCP/IP. The network 106 includes one or more interfaces for data transfer. In some embodiments, the network 106 includes a wired or wireless networking device (not shown) operable to facilitate one or more types of wired and wireless communication between the first electronic device 104, the storage medium 110, as well as other electronic devices (not shown) communicatively connected to the network 106. Examples of the networking device include, but are not limited to, wired and wireless routers, wired and wireless modems, access points, as well as other types of suitable networking devices described herein. Examples of wired and wireless communication include, Ethernet, WiFi, Cellular, LTE, GPS, Bluetooth, RFID, as well as other types of communication modes described herein. In some embodiments, the caregiver 103 and/or another authorized individual may utilize the first electronic device 104 to transmit data indicative of the condition of the patient 102, data indicative of stimuli provided to the patient 102, data indicative of the patient's response to stimuli, as well as other similar data over the network 106 to another electronic device (not shown) that is communicatively connected to the network 106.

The first electronic device 104 is operable to receive data indicative of a current condition of the patient 102. In some embodiments, the current condition of the patient 102 is detected by one or more sensors 105 that are communicatively connected to the first electronic device 104. Moreover, the sensors 105 are operable to detect one or more physical and/or psychological conditions of the patient 102. The sensors 105 may include thermometers, heartrate sensors, blood pressure sensors, oxygen level sensors, digital scales, nano-sensors, brainwave sensors, as well as other sensors that are operable to detect one or more physical and/or psychological conditions of the patient 102. In another one of such embodiments, the current condition of the patient 102 is determined by the caregiver 103 and is manually provided to the first electronic device 104.

The first electronic device 104, upon receipt of the current condition of the patient 102, accesses the storage medium 110 for prior patient data of the patient 102. As stated herein, the prior patient data includes data indicative of stimuli previously experienced by the patient 102 as well as data indicative of how the patient 102 responded to said stimuli. The first electronic device 104 queries the prior patient data to identify a prior condition of the patient (“a prior condition”) that is similar and/or identical to the current condition. Whether a prior condition is similar to the current condition may be predetermined by the caregiver 103. For example, the caregiver 103 may have predetermined that a prior condition where the patient was visibly distressed, lacked appetite for food, and had a heart rate within a range of 65-75 (hereafter referred to as “the first prior condition”) would be considered similar to a current condition where the patient is visibly distressed, lack appetite for food, and has a heart rate of 70. Further, whether a prior condition is similar to a current condition may also be adjusted by the caregiver 103 or dynamically modified by the first electronic device 104. In some embodiments, the first electronic device 104 provides a list of one or more prior patient conditions for display and the caregiver 103 may select a prior condition the caregiver 103 considers is most similar to the current condition of the patient 102.

Once the first electronic device 104 identifies a prior condition, the first electronic device 104, assesses the prior patient data to determine a first stimulus that triggered a positive response from the patient 102 while the patient 102 experienced the prior condition. As stated herein, a stimulus may be associated with a textual, image, audio, visual, audiovisual, multimedia content the patient 102 has previously experienced, an activity the patient 102 has previously participated in, or another type of content, event, and/or action the patient 102 has previously experienced.

A positive response from the patient 102 may be one or more responses described herein that immediately improves the current condition of the patient 102, improves the condition of the patient 102 over a short duration, and/or improves the condition of the patient 102 over a long duration. Continuing with the previous example, the first electronic device 104 may determine that when a picture of the patient's newborn son (hereafter referred to as “the first stimulus”) was provided to the patient 102 while the patient 102 experienced the first prior condition, the first stimulus caused the patient to become less distressed(immediate positive response). Further, the patient 102 became more socially engaging with the caregiver 103 over the next hour (positive response over a short term).

In some embodiments, the first electronic device 104 also accesses the behavior database of the storage medium 110 to determine whether other patients have also experienced the first prior condition, which stimuli were presented to the other patients, as well as how the other patients responded to the stimuli. Continuing with the previous example, the first electronic device 104 determines that 100 other patients have experienced the first prior condition, that a video of a family member (hereafter referred to as the “first universal stimulus”) was presented to the 100 patients while said 100 patients experienced the first prior condition, and that the first universal stimulus triggered a positive reaction from 85 patients, which is a positive response rate of 85%. The first electronic device 104 then determines whether the first universal stimulus triggered a positive response at a rate that is greater than a threshold value and generates a recommendation to provide the first universal stimulus to the patient 102 if the positive patient response rate is greater than the threshold value. The first electronic device 104, upon determining the patient's response to the first universal stimulus, provides the patient's response to the behavior databank 112, where the behavior databank 112 is updated to include the patient's response.

In some embodiments, an identity of the caregiver who provides the stimulus to the patient 102 affects the response of the patient 102. For example, presenting the patient with the first stimulus may not trigger a response from the patient 102 if the picture if the first stimulus is presented by a caregiver who is not related to the patient 102. As such, the first electronic device 104 would not select the first stimulus if the current caregiver 103 is not related to the patient 102.

In some embodiments, multiple different stimuli triggered positive responses from the patient 102 while the patient 102 experienced the first prior condition. In one of such embodiments, the first electronic device 104 selects a stimulus that triggered the most positive response from the patient 102 while the patient 102 experienced the first prior condition. For example, the patient 102 was once presented a picture of a non-relative of the patient 102 (hereafter referred to as “the second stimulus”) while the patient 102 experienced the first prior condition. Although the second stimulus caused the patient to become less distressed, the patient 102 did not become more socially engaging in response to the second stimulus. As such, the first stimulus triggered a more positive reaction relative to the second stimulus, and is thus selected by the first electronic device 104.

The first electronic device 104, upon selecting a stimulus, such as the first stimulus, provides a recommendation for the patient 102 to experience the stimulus, where the recommendation together with the first stimulus are displayed on a display screen of the first electronic device 104. In some embodiments, where the stimulus is associated with content, the content is provided for display on a display screen of the first electronic device 104. In other embodiments, where the stimulus is associated with an activity, instructions regarding how the patient 102 should engage in such activity, instructions regarding how the caregiver 103 should provide such activity, as well as other instructions regarding the activity and the one or more participants of the activity are provided for display on the display screen. Additional information, such as instructions on when the stimulus should be presented to the patient 102, how the stimulus should be presented to the patient 102, who should present the stimulus to the patient 102, one or more alternative stimuli that may be provided to the patient 102, as well as other instructions for providing the stimulus may also be provided for display on the display screen of the first electronic device 104 to help the caregiver 103 better present the stimulus.

The stimulus triggers a response from the patient 102. As defined herein, the patient's response may include a positive response, a negative response, a neutral response, or a lack of a response. In some embodiments, the patient's response is picked up by the sensors 105 and is transmitted by the sensors 105 to the first electronic device 104. In other embodiments, the caregiver 103 manually provides the patient's response to the first electronic device 104. In further embodiments, sensor components (not shown) of the first electronic device 104 are operable to detect and obtain the patient's response. The first electronic device 104, upon receipt of the patient's response, stores data indicative of the response on the storage medium 110, where the response becomes a part of the patient's prior history that may be used to determine which stimulus should be provided to the patient 102 in the future. The first electronic device 104 may categorize the response as a positive response, a negative response, a neutral response, or a lack of response. Moreover, the first electronic device 104 may arrange data indicative of one of the patient's responses based on one of the foregoing categories.

The electronic device 104 may continue to provide additional stimuli to the patient 102 to further improve the patient's current condition. Continuing with the previous example, the first stimulus to the patient 102 triggered a positive response from the patient 102, and improved the patient's current condition from visibly distressed to not visibly distressed and from being not socially engaging to being more socially engaging. However, the patient's current condition still indicates that the patient lacks appetite for food. The first electronic device 104 assesses the prior patient data for a prior condition that is similar and/or identical to the current condition. The first electronic device 104 identifies a prior condition where the patient 102 was not visibly distressed, was socially engaging, lacked appetite, and had a heart rate between 62-77 (hereafter referred to as the “second prior condition”), and determines that the second prior condition is similar and/or identical to the current condition of the patient.

The first electronic device 104 then assesses the prior patient data to determine a stimulus that triggered a positive response from the patient 102 while the patient 102 experienced the second prior condition. Continuing with the previous example, the first electronic device 104 determines that when the caregiver 103 engaged in a conversation with the patient 102 about sushi and showed the patient 102 images of sashimi (hereafter collectively referred to as “the third stimulus”) while the patient 102 experienced the second prior condition, the patient's gained appetite and expressed interest in having a meal. The first electronic device 104 then provides a recommendation for the patient to experience the third stimulus, where the third stimulus together with instructions on how to present the third stimulus are provided for display on the display screen of the first electronic device 104. The patient's response to the third stimulus is detected by the sensors 105, inputted by the caregiver 103, and/or detected by sensor components of the first electronic device 104. In some embodiments, the foregoing process for providing additional stimuli may continue for a predetermined number of iterations. In further embodiments, the foregoing process for providing additional stimuli may be periodically performed by the first electronic device 104.

In some embodiments, the first electronic device 104 analyzes each stimulus provided to the patient 102 and each corresponding response triggered by the stimulus to identify a set of parameters that define how to present the stimulus. Parameters that define how to present the stimulus include how the stimulus was previously presented to the patient 102, how the patient 102 experienced the stimulus, the identity of an individual that appears in the stimulus, the date and time the stimulus was presented to the patient 102, the number of times the stimulus has been presented to the patient 102 over an operational duration, the total number stimuli that have been presented to the patient 102 over the duration, as well as other quantifiable parameters.

In one of such embodiments, where the patient 102 experienced the first prior condition on two separate occasions, and the first stimulus triggered two different responses from the patient 102 when the first stimulus was presented on the two occasions, the first electronic device 104 is operable to assess the parameters that define how to present first stimulus to determine whether the parameters may be modified to improve the patient's response. In one example, one parameter that defines how to present the first stimulus stipulates that that the first stimulus may be presented to the patient 102 in the morning and the afternoon. However, presenting the first stimulus in the morning caused the patient's heart rate to increase whereas presenting the first stimulus in the afternoon had no effect on the patient's heart rate. Based on the foregoing, the first electronic device 104 determines that the first stimulus should not be presented in the morning and modifies the parameters accordingly.

In another one of such embodiments, the first electronic device 104 is operable to compare one or more parameters that define how to present the first stimulus with one or more parameters that define how to present similar stimuli, such as the second stimulus. In one example, one parameter that defines how to present stimuli to the patient 102 is based on an identity of an individual that appears in a stimulus. Assuming that all other factors are identical, the first electronic device 104 determines that the patient 102 experienced a more positive response when the patient 102 viewed pictures of the patient's newborn instead of pictures of non-related individuals. The first electronic device 104 may recommend that he patient 102 should also present similar stimuli (e.g., stimuli associated with presenting photographs of the spouse, photographs of other children, photographs of family members, as well as other similar stimuli) to trigger a more positive response from the patient 102.

In another one of such embodiments, the first electronic device 104 determines how the parameters cause a stimulus, or similar stimuli to trigger different responses from the patient 102. The first electronic device 104 then formulates a new stimulus based on the stimulus and/or similar stimuli and based on one or more parameters that are associated with the stimulus and/or similar stimuli. For example, if stimuli associated with pictures of the patient's family members triggered positive responses, the first electronic device 104 may determine to formulate a new stimulus associated with a video recording of the patient's family members. The first electronic device 104 may then associate one or more parameters that define how to present stimuli associated with pictures of the patient's family members with the new stimulus.

In a further one of such embodiments, the first electronic device 104 is operable to compare parameters that define how to present stimuli that triggered negative reactions and/or neutral reactions with parameters that define how to present with similar stimuli that triggered positive reactions. The first electronic device 104 is further operable to determine whether the parameters associated with stimuli that triggered negative and/or neutral reactions may be modified to cause the stimuli to trigger positive reactions. For example, if the first electronic device 104 determines that a stimulus associated with presenting the patient 102 with a video recording of the patient's family members (hereafter referred to as “the fourth stimulus”) increases the patient's blood pressure, the first electronic device 104 may assess stimuli associated with presenting other video recordings of the patient's family members that did not increase the patient's blood pressure. The first electronic device 104 then determines that the fourth stimulus was presented to the patient 102 after 8:00 pm whereas stimuli similar to the fourth stimulus were presented to patient 102 before 2:00 pm. The first electronic device 104 then modifies a parameter that defines when the fourth stimulus may be presented to limit presentation time of the fourth stimulus.

In some embodiment, when the first electronic device 104 is presented with new patient conditions, the first electronic device 104 conducts a behavior analysis based on data stored in the behavior database to identify one or more stimuli that has a high probability to trigger a positive response, and provides a recommendation of the one or more stimuli to the caregiver 103. For example, If the patient 102 experiences a new condition where the patient is upset, refuses to communicate with the caregiver 103, and has no appetite, the first electronic device 104 may access the behavior database to determine one or more universal stimuli that has a probability above a threshold value to trigger a positive response from someone who is upset, refuses to communicate, and has no appetite. The first electronic device 104 then recommends the universal stimuli to the caregiver 103. The first electronic device 104 is also operable to determine the patient's response to the universal stimuli and to determine whether to recommend the universal stimuli in the future based on how the patient 102 responds to the universal stimuli. As such, the first electronic device 104 is operable to continuously and dynamically evolve to learn from prior and current responses from the patient to predict stimuli and provide stimuli that trigger positive experiences to improve the current condition of the patient 102.

In some embodiments, the first electronic device 104 is operable to generate a timeline of prior conditions of the patient 102, stimuli presented to patient 102, as well as the patient's responses to the stimuli. The timeline may be accessible by the caregiver 103 and/or other authorized individuals to monitor the progress of the patient 102. In some embodiments, the first electronic device 104 includes additional hardware and software components that are operable to allow the caregiver 103 and/or other authorized individuals to remotely access the first electronic device 104 to monitor the patient 102 and to remotely provide different stimuli to the patient 102.

Although the foregoing operations are described to be performed by the first electronic device 104, the foregoing operations may be performed by another electronic device that is operable to obtain data indicative of the condition of the patient 102, determine which stimulus should be presented to the patient 102, and to provide a recommendation for the patient 102 to experience said stimulus. In some embodiments, the foregoing operations are performed by an application running on a remote electronic device (not shown) that is stationed at a remote location relative to the location of the patient 102. In such embodiments, the remote electronic device provides recommendations over the network 106 to the first electronic device 104, where the recommendations and the stimuli associated with the recommendations are provided for display on the first electronic device 104. Although FIG. 1 illustrates a single electronic device 104 connected to the storage medium 110 via the network 106, additional electronic devices (not shown) may also be communicatively connected to the first electronic device 104 via the network 106.

FIG. 2 is a flow chart that illustrates a process to provide positive stimuli to a patient suffering from an in accordance with one embodiment. Although the paragraphs below describe the operations of process 200 being performed by the first electronic device 104 illustrated in FIG. 1, the instructions for the operations may be stored locally on the first electronic device 104 or may be stored remotely, such as on the storage medium 110. Further, the process may be performed by other electronic devices described herein. Further, although operations in the process 200 are shown in a particular order, certain operations may be performed in different orders or at the same time where feasible.

At step 202, the first electronic device 104 obtains data indicative of a current condition of the patient 102. As stated herein, data indicative of the current condition may be obtained from the sensors 105, from the caregiver 103, or directly by the first electronic device 104. As step 204, the first electronic device 104 determines a stimulus that previously triggered a positive reaction from the patient while the patient was in a condition similar and/or identical to the current condition. Operations to determine a positive stimulus are described in detail in the foregoing paragraphs. At step 206, the first electronic device provides a recommendation for the patient 102 to experience the stimulus. In some embodiments, the stimulus is automatically provided for display on the display screen of the first electronic device 104. In other embodiments, the caregiver 103 determines whether to provide the stimulus to the patient 102. In further embodiments, instructions regarding how to present the stimulus may also be included in the recommendation. At step 208, the first electronic device determines whether data indicative of the patient's response to the stimulus has been received. If the first electronic device does not receive a response within an operational duration (e.g., ten seconds, one minute, or another quantifiable operational duration), then the process proceeds to step 214, which is described in the paragraphs below.

Alternatively, if the first electronic device 104 receives a response from the patient within the operational duration, then the process proceeds to step 210 and the first electronic device 104 stores data indicative of the first response on the storage medium 110 or another storage medium that contains the patient's data. At step 212, the first electronic device 104 determines if patient's current condition has changed. If the patient's current condition has changed, then the process proceeds to step 218 and the first electronic device 104 determines whether data indicative of the patient's new condition should be obtained and analyzed. If the first electronic device 104 determines that the patient's new condition should be obtained and analyzed, then the process returns to step 202, data indicative of the patient's new condition are obtained, and the process described in the foregoing paragraphs is repeated. Alternatively, if the first electronic device 104 receives instructions from the caregiver 103 or is preprogramed not to obtain data indicative of the new condition, then the process ends at step 220.

Alternatively, if the first electronic device 104 determines at step 212 that the patient's condition has not changed, then the process proceeds to 214. At step 214, the first electronic device 104 determines a different stimulus that previously triggered a positive reaction from the patient while the patient was in a condition similar to the current condition. At step 216, the first electronic device 104 determines whether to recommend the different stimulus to the patient 102. If the first electronic device 104 determines that the different stimulus should not be recommended to the patient 102, or if the first electronic device 104 receives instructions from the caregiver 103 not to recommend the different stimulus, then the process ends at step 220. Alternatively, if the first electronic device 104 determines to provide the different stimulus to the patient 102, then the process returns to step 206 and the process described in the foregoing paragraphs is repeated.

FIG. 3A is a flow chart that illustrates a process to dynamically formulate new stimuli based on the patient's responses to stimuli in accordance with one embodiment. Although the paragraphs below describe the operations of process 300 being performed by the first electronic device 104 illustrated in FIG. 1, the instructions for the operations may be stored locally on the first electronic device 104 or may be stored remotely, such as on the storage medium 110. Further, the process may be performed by other electronic devices described herein. Further, although operations in the process 300 are shown in a particular order, certain operations may be performed in different orders or at the same time where feasible.

At step 302 the first electronic device 104 receives data indicative of the patient's response to a stimulus. As stated herein, the response may be detected, monitored, and/or recorded by one or more sensors, components, and/or electronic devices that are operable to detect, monitor, and/or record the patient's response and further operable to provide data indicative of the patient's response to the first electronic device 104. At step 304, the first electronic device 104 performs operations described herein to determine whether the response corresponds to a positive response. If the first electronic device 104 determines that the response is a positive response, then the process proceeds to step 306 and the first electronic device 104 determines whether to formulate a new stimulus based on the positive response.

In some embodiments, the first electronic device 104, upon determining that the patient's response is a positive response, incorporates the patient's response and a stimulus that triggered the patient's response into a new stimulus that includes both the stimulus and the patient's response. For example, if the patient responds to the first stimulus (a picture of the patient's newborn) by mentioning the newborn's name (hereafter referred to “the first positive response to the first stimulus”), then the first electronic device 104 may obtain, from a video recorder that is communicatively connected to the first electronic device 104, a video recording of the patient's interaction with the first stimulus and the patient's positive response to the first stimulus (the video recording hereafter referred to as “the first new stimulus”). The first electronic device 104 then determines one or more patient conditions (e.g., in response to the first prior condition) to present the first new stimulus.

In other embodiments, the first electronic device 104 formulates a new stimulus solely based on the patient's response to a stimulus. Continuing with the previous example, the first electronic device 104 may formulate a video recording containing only the patient's response to the first stimulus (hereafter referred to as “the second new stimulus”). The first electronic device 104 then determines one or more patient conditions to present the second new stimulus.

In some embodiments, if the first electronic device 104 determines at step 306 that a new stimulus should be formed, the first electronic device 104 also determines one or more of the patient's conditions for recommending the new stimulus to the patient. In some embodiments, the first electronic device 104 identifies one or more of the patient's prior conditions that were improved in response to stimuli similar to the new stimulus, and associates the new stimulus to the patient's said prior conditions. For example, if the first electronic device 104 identifies that the patient's first prior condition was improved in response to the first stimulus, and determines that the first stimulus is similar to the new stimulus, then the first electronic device 104 also associates the new stimulus with the first prior condition, and would recommend the new stimulus to the patient if the patient subsequently experiences the first prior condition. In some embodiments, the new stimulus includes one or more modifications of existing stimuli. In one example, the new stimulus may combine multiple existing stimuli. In another example, the new stimulus may be in a format that is different from the existing stimuli. In a further example, a portion of existing stimuli, such as a portion of a text file, an image, a video recording, a multimedia content, or another type of content disclosed herein may be obscured or censored in the new stimulus.

The process then proceeds to step 308 and the first electronic device 104 stores the new stimulus together with the conditions for recommending the new stimulus on the storage medium 110. The process then proceeds to step 320. Alternatively, if the first electronic device 104 determines not to formulate a new stimulus at step 306, the process also proceeds to step 320. At step 320, the electronic device 104 determines whether to assess data indicative of the patient's subsequent responses to stimulus. The process ends at step 322 if the first electronic device 104 determines not to assess data indicative of additional responses. Alternatively, if the first electronic device 104 determines at step 320 to continue to assess the patient's responses, the process returns to step 302. In some embodiments, the first electronic device 104 has a default setting to continue to assess the patient's subsequent responses until the first electronic device 104 receives overriding instructions from the caregiver 103 or another authorized personnel to cease monitoring the patient's responses.

Alternatively, if the first electronic device 104 determines at step 304 that the response is not a positive response (e.g., the response is a negative response, a neutral response, or constitutes a lack of a response), then the process proceeds to step 314, and the first electronic device 104 performs operations described herein to determine one or more parameters of the existing stimulus that triggered a non-positive response. Examples of parameters may include parameters that quantify how the stimulus was previously presented to the patient, how the patient experienced the stimulus, the identity of an individual that appears in the stimulus the date and time the stimulus was presented to the patient, the number of times the stimulus has been presented to the patient over an operational duration, the total number stimuli that have been presented to the patient over the duration, as well as other quantifiable parameters. At step 316, the first electronic device 104 determines whether to formulate new stimulus based on the one or more parameters determined in step 314, wherein the new stimulus contains one or more modifications of one or more existing stimuli. If the first electronic device 104 determines to formulate the new stimulus, then the first electronic device 104 performs operations described herein to formulate the new stimulus. In one example, a portion of existing stimuli, such as a portion of a text file, an image, a video recording, a multimedia content, or another type of content disclosed herein may be obscured or censored in the new stimulus. In another example, the new stimulus may be in a format that is different from a format of existing stimuli. In a further example, the new stimulus may contain a combination of multiple existing stimuli. The process then proceeds to step 318 and the first electronic device 104 stores the new stimulus on the storage medium 110. The process then proceeds to step 320, which is described in the foregoing paragraph. Moreover, if the first electronic device 104 determines at step 316 not to formulate a new stimulus, then the process also proceeds to step 320. In view of the foregoing, the first electronic device 104 is operable to continuously and dynamically adapt to the patient's current condition by formulating new stimuli based on the patient's reactions to existing stimuli to trigger positive responses from the patient.

In one embodiment, an initial set up of the first electronic device 104 includes storing written material, audio recordings, video recordings, and digital photographs to be used as primary stimulus, corresponding, for example, to life experiences and memory references for a patient. The process of FIG. 3A can be used to generate derivative stimulus. The impact of such derivative stimulus on a patient response can then be evaluated for a positive patient response and potential future use by first electronic device 104. In such a manner, first electronic device 104 can continuously create and evaluate new stimulus to dynamically create a library of stimuli that best improves patient behaviors and outcomes. The recursive nature of the algorithm to create, test, modify, and select such library of stimuli represents one manner in which self-learning technology can be utilized to create a considerable advancement in treatment for patients' suffering, for example, from dementia and other cognitive disorders. Such algorithm also works to customize a treatment plan specific to each patient and how they react to particular stimuli.

The first electronic device 104 described in the above-disclosed embodiments can be configured to store a current “deck” of stimulus and vary the order, frequency, and timing of the presentation of such deck to a patient. Moreover, the first electronic device 104 may monitor the patient's response to such deck on a real-time basis, or, alternatively, on a batched basis (once a day or once a week, for example), and the order of the deck shuffled or the stimulus included in such deck modified based on such monitoring. For example, in a real-time monitoring example, the first electronic device 104 maintains an overall patient condition score that may be modified upon the presentation of each stimulus. If the first electronic device 104 determines that a stimulus causes a material reduction in the overall patient condition score, the first electronic device 104 may immediately remove the stimulus from a deck. If the overall patient condition score is not improving, the deck may be shuffled to be presented in a different order, or a different set of stimulus can be used to create a new deck. In one embodiment, different patient condition categories may have different scores. For example, one score may be for patient memory, one for patient distress, one for patient disorientation, and one for patient mood. The scores for each category may carry the same weight. Alternatively, the scores may be weighted based on category, where one the score associated with one category (e.g., patient disorientation) is given greater value relative to the score associated with another category (e.g., patient distress). A caregiver or an authorized personnel may adjust the weight of the scores based on a patient by patient basis. Alternatively, the first electronic device 104 may dynamically adjust the weight of the scores based on a combination of the patient's conditions, stimuli presented to the patient, and the patient's response to said stimuli. For example, if the patient persistently experiences anxiety over an operational duration (e.g., one hour, one day, or another quantifiable period of time), then the first electronic device 104 increases the weight of the score associated with anxiety and reshuffle and/or create a new deck of stimuli more tailored to reduce the patient's anxiety. Portions of the deck may be directed to different patient condition categories depending on a particular patient's malady, symptoms, disease progression, and responsiveness to treatment. In some embodiments, the deck and instructions for modifying the deck and creating the deck are stored on the first electronic device 104. In other embodiments, the deck and the instructions are stored on the storage medium 110 or another device (not shown) that is accessible to the first electronic device 104 via the network 106.

From the foregoing embodiments, the system and behavior databank 112 can also be used to store, compile and analyze data that may be valuable for researching further treatment, particularly when combined with demographic information, malady information, disease progression information, symptoms or other information. Personal information may be removed in accordance with any applicable regulatory, legal, and ethical standards. Audio or video recordings of patient responses can be automatically translated into a data record to remove personal information and summarize such recordings with metadata. For example, a video of a particular patient may be translated by first electronic device 104 into a flat searchable database record for behavior databank 112 that includes patient sex, patient age, malady, progression, symptoms, stimulus applied, and response observed (which may include a textual description of observed patient behavior or a measured condition such as particular neural feedback or brainwave activity). An algorithm may be used by doctors or researchers to select relevant research criteria and analyze aggregated responses to particular stimulus categories. For example, if a memory of a traumatic event (a combat experience) is triggered by a particular sound (gunfire, for example) such that a patient becomes distraught, a researcher may use the system to determine which stimulus have been introduced in similar situations (while paired with the sound or introduced immediately following the sound) that have either disassociated the sound with the memory of the traumatic event or lessened the distraught response of a patient. Over time, statistical, aggregated information can be stored in or derived from behavior databank 112 to be accessible to caregivers, clinicians, doctors, psychologists, scientists, and researchers. If a stimulus in a particular category or determined to include particular characteristics has been shown to have no benefit or a negative benefit with respect to a particular patient symptom or condition, the system may not allow such stimulus to be presented to a patient, or may alternatively be flagged or an alert generated with such stimulus is selected for presentation to a patient. Such feature would be an important source of preventing human error leading to situations that may result in severe patient distress.

FIG. 3B is a flow chart that illustrates a process 350 to dynamically formulate new stimuli based on the patient's responses to stimuli in accordance with another embodiment. Steps 304, 306, 308, 314, 316, and 318 of the process 350 are identical to corresponding steps of the process 300 of FIG. 3A. However, steps 304, 306, 308, 314, 316, and 318 of the process 350 runs one iteration, whereas the corresponding steps of the process 300 may loop back and repeat. In some embodiments, the process 350 would be implemented as a sub-process of the process 200 as an alternative to step 210. Moreover, the steps of process 350 begin after the first electronic device 104 receives a response triggered by the first stimulus at step 208. Further, at step 320, the process 350 proceeds to step 212 of the process 200.

As used in this specification and any claims of this application, the terms “computer”, “server”, “processor”, and “memory” all refer to electronic or other technological devices. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

The above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification.

The above disclosed embodiments have been presented for purposes of illustration and to enable one of ordinary skill in the art to practice the disclosed embodiments, but is not intended to be exhaustive or limited to the forms disclosed. Many insubstantial modifications and variations will be apparent to those of ordinary skill in the art without departing from the scope and spirit of the disclosure. For instance, although the flowcharts depict a serial process, some of the steps/blocks may be performed in parallel or out of sequence, or combined into a single step/block. The scope of the claims is intended to broadly cover the disclosed embodiments and any such modification.

As used herein, the singular forms “a”, “an” and “the” are intended to include the plural forms as well, unless the context clearly indicates otherwise. It will be further understood that the terms “comprise” and/or “comprising,” when used in this specification and/or the claims, specify the presence of stated features, steps, operations, elements, and/or components, but do not preclude the presence or addition of one or more other features, steps, operations, elements, components, and/or groups thereof. In addition, the steps and components described in the above embodiments and figures are merely illustrative and do not imply that any particular step or component is a requirement of a claimed embodiment.

1. A computer-implemented method to provide a patient with positive stimulus, the method comprising:
obtaining data indicative of a current condition of a first patient;
determining, based on prior patient data of the first patient, a first stimulus that triggered a positive response from the first patient while the first patient was in a condition similar to the current condition;
providing a first recommendation for the first patient to experience the first stimulus for display on an electronic device;
receiving a first response triggered by the first stimulus; and
storing data indicative of the first response in a storage medium.

2. The computer-implemented method of claim 1, further comprising:
obtaining data indicative of a new condition of the first patient, the new condition at least partly attributed to the first stimulus and the first response;
determining, based on the prior patient data of the first patient, a second stimulus that triggered a positive response from the first patient while the first patient was in a condition similar to the new condition; and
providing a second recommendation for the first patient to experience the second stimulus for display on the electronic device.
receiving a second response triggered by the second stimulus; and
storing data indicative of the second response in a storage medium.

3. The computer-implemented method of claim 1, further comprising determining whether the first response corresponds to a positive response, a negative response, or a neutral response.

4. The computer-implemented method of claim 3, further comprising:
identifying a first set of parameters that caused the first stimulus to trigger the first response; and
formulating, based on the first set of parameters, a third stimulus projected to trigger a positive response while the first patient is in the current condition.

5. The computer-implemented method of claim 3, further comprising:
identifying a first set of parameters that define how to present the first stimulus to the first patient; and
modifying one or more of the first set of parameters to improve the first response.

6. The computer-implemented method of claim 3, further comprising:
indicating the first stimulus as a stimulus that triggered a positive response from the first patient while the first patient is in the current condition,
wherein the first stimulus is recommended to the first patient if the first patient is in a condition that is similar to the current condition.

7. The computer-implemented method of claim 6, further comprising:
indicating the first stimulus as a stimulus that triggered a negative response from the first patient while the first patient is in the current condition,
wherein the first stimulus is not be recommended to the first patient if the first patient is in a condition that is similar to the current condition.

8. The computer-implemented method of claim 1, wherein the first stimulus is associated with at least one of a text and an image the first patient has previously encountered, and wherein the computer-implemented method further comprises providing the at least one of the text and the image of the first patient for display on the electronic device.

9. The computer-implemented method of claim 1, wherein the first stimulus is associated with at least one of an audio recording, a video recording, an audiovisual recording the first patient has previously experienced, and wherein the computer-implemented method further comprises providing the at least one of the audio recording, the video recording, and the audiovisual recording to the electronic device.

10. The computer-implemented method of claim 1, wherein the first stimulus is associated with an activity the first patient has previously participated in, and wherein the computer-implemented method further comprises providing instructions on how the first patient should participate in the activity for display on the electronic device.

11. The computer-implemented method of claim 1, further comprising:
determining a cause of at least one communication and memory impairment the first patient suffers from,
wherein determining the first stimulus is further based on the cause of neurodegenerative condition a patient suffers from.

12. The computer-implemented method of claim 1, further comprising:
identifying an individual that appears in the first stimulus and a relationship between the individual and the first patient,
wherein determining the first stimulus is further based on at least one of the identity of the individual that appears in the first stimulus and the relationship between the individual and the first patient.

13. The computer-implemented method of claim 1, further comprising:
formulating a new stimulus based on the first response, wherein the new stimulus comprises the first response; and
storing the new stimulus on the storage medium.

14. The computer-implemented method of claim 1, wherein obtaining data indicative of the current condition of the first patient comprises receiving, from a sensor operable to detect at least one of a physical or a psychological condition of the first patient.

15. The computer-implemented method of claim 11, where the at least one communication and memory impairment of the first patient is caused by at least one of Dementia, Alzheimer's, and PTSD, Traumatic Brian Injury, Stroke, Chronic Traumatic Encephalopathy, and Autism, and further comprising categorizing the first response based on the cause of the at least one communication and memory impairment.

16. An electronic device for alleviating at least one communication and memory impairment, the electronic device comprising:
a processor operable to:
obtain data indicative of a current condition of a first patient;
determine, based on prior patient data of the first patient, a first stimulus that triggered a positive response from the first patient while the first patient was in a condition similar to the current condition;
receive a first response triggered by the first stimulus; and
store data indicative of the first response in a storage medium; and
a display screen operable to provide a first recommendation for the first patient to experience the first stimulus for display.

17. A method to alleviate a neurodegenerative condition, the method comprising:
determining a cause of a neurodegenerative condition a first patient suffers from;
obtaining a current condition of the first patient;
determining, based on the cause of the neurodegenerative condition the patient suffers from and prior patient data of the first patient, a first stimulus that triggered a positive response from the first patient while the first patient was in a condition similar to the current condition;
providing a first recommendation for the first patient to experience the first stimulus;
receiving a first response triggered by the first stimulus; and
augmenting the prior data of the first patient to include data indicative of the first response.
